(19) United States
(12) Patent Application Publication
(10) Pub. No.: US 2025/0182348 A1
(43) Pub. Date: Jun. 5, 2025

(54) MEDICAL INFORMATION PROCESSING APPARATUS, METHOD, AND STORAGE MEDIUM

(71) Applicant: Canon Medical Systems Corporation, Otawara-shi (JP)

(72) Inventor: Shinsuke TSUKAGOSHI, Nasushiobara (JP)

(73) Assignee: Canon Medical Systems Corporation, Otawara-shi (JP)

(21) Appl. No.: 18/969,244

(22) Filed: Dec. 4, 2024

(30) Foreign Application Priority Data
Dec. 5, 2023 (JP) 2023-205352

(51) Int. Cl. G06T 11/00; A61B 6/00; A61B 6/03; A61B 6/42; A61B 6/46

(52) U.S. Cl. CPC G06T 11/008; A61B 6/032; A61B 6/463; A61B 6/5294; A61B 6/4241; G06T 2210/41

(57) ABSTRACT

According to one embodiment, a processing circuitry acquires count data related to a plurality of energy bins collected in a PCCT scan. The processing circuitry reconstructs PCCT images based on count data related to a first energy bin. The processing circuitry generates utilization information for evaluating a utilization degree of X-ray energy of X-rays emitted in the PCCT scan for reconstruction of the PCCT images based on the first energy bin used to reconstruct the PCCT images or a second energy bin not used to reconstruct the PCCT images. The processing circuitry stores the utilization information in a storage device in association with the PCCT images and/or related information of the PCCT images.

### CROSS-REFERENCE TO RELATED APPLICATIONS

This application is based upon and claims the benefit of priority from Japanese Patent Application No. 2023-205352, filed Dec. 5, 2023, the entire contents of which are incorporated herein by reference.

### FIELD

Embodiments described herein relate generally to medical information processing apparatus, a method, and a storage medium.

### BACKGROUND

Photon counting computed tomography (PCCT) can generate images of various purposes by performing data collection in a plurality of energy bins. For example, in a case where an image is generated only from count data of a specific energy bin or in a case where an image is generated from count data of an energy bin other than the specific energy bin, only a part of the energy of the X-rays emitted to the subject and detected by the X-ray detector contributes to imaging.

### BRIEF DESCRIPTION OF THE DRAWINGS

FIG. 1 is a diagram showing a configuration of an X-ray computed tomography apparatus according to the present embodiment.

FIG. 2 is a view showing an example of a process procedure of a PCCT examination by the X-ray computed tomography apparatus.

FIG. 3 is a diagram showing an example of setting bin setting information.

FIG. 4 is a conceptual diagram of saving a PCCT image and energy utilization information in a data storage device.

FIG. 5 is a diagram showing an example of a display screen of a PCCT image, energy utilization information, and a determination result according to step S7.

FIG. 6 is a diagram showing an example of a display screen of a PCCT image, energy utilization information, and a determination result according to step S8.

FIG. 7 is a diagram showing another example of a display screen of a PCCT image, energy utilization information, and a determination result according to step S8.

FIG. 8 is a conceptual diagram of saving a comment in a data storage device.

FIG. 9 is a conceptual diagram of saving energy utilization information of a plurality of PCCT images in a data storage device.

### DETAILED DESCRIPTION

In general, according to one embodiment, a medical information processing apparatus includes an acquisition unit, a reconstruction unit, a generation unit, and a storage unit. The acquisition unit acquires count data related to a plurality of energy bins collected in a PCCT scan on a subject. The reconstruction unit reconstructs one or a plurality of PCCT images based on count data related to a first energy bin among the plurality of energy bins. The generation unit generates utilization information for evaluating a utilization degree of X-ray energy of an X-ray emitted in the PCCT scan for reconstruction of the one or plurality of PCCT images based on the first energy bin used to reconstruct the one or plurality of PCCT images or a second energy bin not used to reconstruct the one or plurality of PCCT images other than the first energy bin of the plurality of energy bins. The storage unit stores the utilization information in a storage device in association with the one or more PCCT images and/or related information of the one or plurality of PCCT images.

Hereinafter, medical information processing apparatus, a method, and a program according to the present embodiment will be described in detail with reference to the drawings.

The X-ray computed tomography apparatus according to the present embodiment includes various types such as a third generation CT and a fourth generation CT, and any type is applicable to the present embodiment. Here, the third generation CT is a Rotate/Rotate-Type in which the X-ray tube and the X-ray detector integrally rotate around the subject. The fourth generation CT is a Stationary/Rotate-Type in which a large number of X-ray detection elements arranged in a ring shape are fixed, and only an X-ray tube rotates around a subject. In addition, the X-ray computed tomography apparatus according to the present embodiment can be applied to a single tube type in which one pair of an X-ray tube and an X-ray detector is mounted on a rotation ring, or a multi-tube type in which a plurality of pairs of an X-ray tube and an X-ray detector is mounted on a rotation ring. However, in the following description, the X-ray computed tomography apparatus is assumed to be a single tube type.

The X-ray computed tomography apparatus according to the present embodiment is assumed to be a photon counting CT apparatus that performs photon counting computed tomography (PCCT).

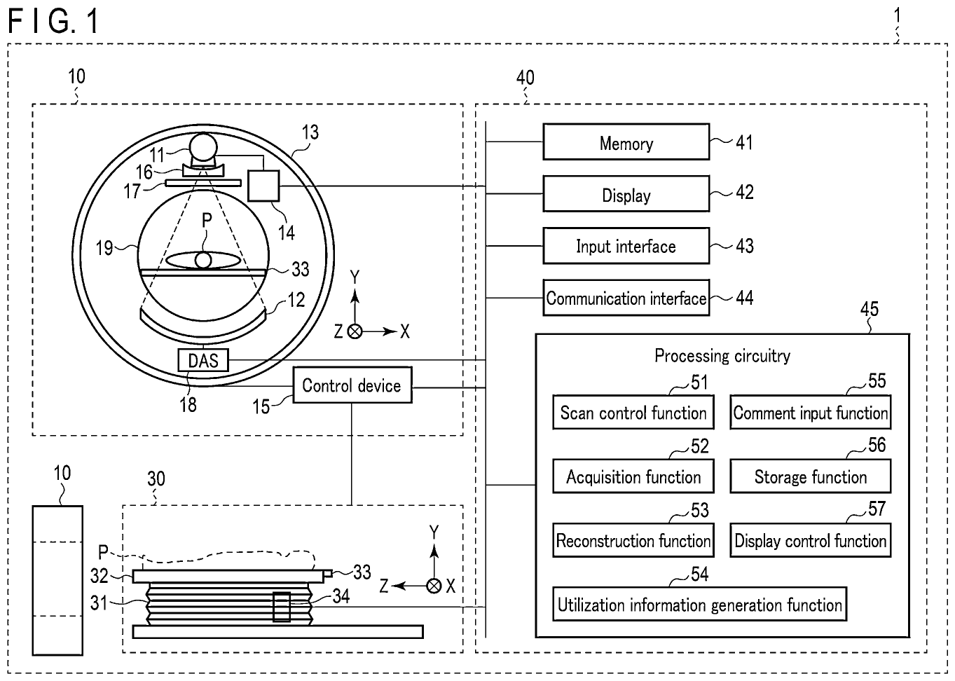

FIG. 1 is a diagram showing a configuration of an X-ray computed tomography apparatus 1 according to the present embodiment. As shown in FIG. 1, the X-ray computed tomography apparatus 1 includes a gantry 10, a couch 30, and a console 40. Note that, in FIG. 1, the gantry 10 is shown at a plurality of locations for convenience of description, but one stand 10 or a plurality of stands may be mounted on the X-ray computed tomography apparatus 1. The gantry 10 is a scanning device having a configuration for PCCT scanning of a subject P. The couch 30 is a transfer device for placing the subject P to be subjected to the X-ray CT imaging and positioning the subject P. The console 40 is a computer that controls the gantry 10. For example, the gantry 10 and the couch 30 are installed in a CT examination room, and the console 40 is installed in a control room adjacent to the CT examination room. The gantry 10, the couch 30, and the console 40 are connected in a wired or wireless manner so as to be able to communicate with each other. The console 40 is not necessarily installed in the control room. For example, the console 40 may be installed in the same room together with the gantry 10 and the couch 30. The console 40 may also be incorporated into the gantry 10. The console 40 is an example of the medical information processing apparatus.

As shown in FIG. 1, the gantry 10 includes an X-ray tube 11, an X-ray detector 12, a rotary frame 13, an X-ray high-voltage device 14, a control device 15, a wedge 16, a collimator 17, and a data acquisition system (DAS) 18.

The X-ray tube 11 generates an X-ray. Specifically, the X-ray tube 11 includes a cathode that generates thermoelectrons, an anode that receives the thermoelectrons flying from the cathode and generates X-rays, and a vacuum tube that holds the cathode and the anode. The X-ray tube 11 is connected to the X-ray high-voltage device 14 through a high voltage cable. A tube voltage is applied between the cathode and the anode by the X-ray high-voltage device 14. The application of the tube voltage causes thermoelectrons to fly from the cathode toward the anode. If thermoelectrons fly from the cathode toward the anode, a tube current flows. By application of a high voltage and supply of a filament current from the X-ray high-voltage device 14, thermoelectrons fly from the cathode (filament) toward the anode (target), and the thermoelectrons collide with the anode, thereby generating X-rays. For example, the X-ray tube 11 is a rotating anode type X-ray tube that generates X-rays by irradiating a rotating anode with thermoelectrons.

The X-ray detector 12 detects the X-rays generated from the X-ray tube 11 and passing through the subject P, and outputs an electric signal corresponding to the energy of the detected X-rays to the data acquisition system 18. The X-ray detector 12 has a structure in which a plurality of X-ray detection element arrays in which a plurality of X-ray detection elements are arrayed in the channel direction are arrayed in the slice direction (column direction). The X-ray detector 12 is, for example, an indirect conversion type detector having a grid, a scintillator array, and an optical sensor array. The scintillator array includes a plurality of scintillators. The scintillator generates a plurality of fluorescent photons according to the energy of incident X-ray photons. The grid includes an X-ray shielding plate that is disposed on the X-ray incident surface side of the scintillator array and absorbs scattered X-rays. Note that the grid may also be referred to as a collimator (a one-dimensional collimator or two-dimensional collimator). The optical sensor array converts the plurality of fluorescent photons from the scintillator into an electrical signal having a peak value corresponding to the energy of the incident X-ray photons. As the optical sensor, for example, a photodiode is used.

The X-ray detector 12 may be a direct-conversion type detector. As the direct-conversion type X-ray detector 12, for example, a type including a semiconductor diode in which electrodes are attached to both ends of a semiconductor is applicable. The X-ray photons incident on the semiconductor are converted into electron-hole pairs. The number of electron-hole pairs generated by incidence of one X-ray photon depends on the energy of the incident X-ray photon. The electrons and the holes are attracted to each other by a pair of electrodes formed at both ends of the semiconductor. The pair of electrons generate electrical signals having a peak value corresponding to the charge of the electron-hole pairs. One electrical signal includes a peak value corresponding to the energy of the incident X-ray photon.

The rotary frame 13 is an annular frame that rotatably supports the X-ray tube 11 and the X-ray detector 12 about a rotation axis (Z axis). Specifically, the rotary frame 13 supports the X-ray tube 11 and the X-ray detector 12 facing each other. The rotary frame 13 is supported by a fixed frame (not shown) so as to be rotatable about a rotation axis. The control device 15 rotates the rotary frame 13 about the rotation axis to rotate the X-ray tube 11 and the X-ray detector 12 about the rotation axis. The rotary frame 13 receives power from the drive mechanism of the control device 15 and rotates around the rotation axis at a constant angular velocity. An image field of view (FOV) is set in an opening 19 of the rotary frame 13.

In the present embodiment, the longitudinal direction of the rotation axis of the rotary frame 13 or a top plate 33 of the couch 30 in the non-tilting state is defined as the Z-axis direction, the axial direction orthogonal to the Z-axis direction and horizontal to the floor surface is defined as the X-axis direction, and the axial direction orthogonal to the Z-axis direction and vertical to the floor surface is defined as the Y-axis direction.

The X-ray high-voltage device 14 includes a high-voltage generator and an X-ray controller. The high voltage generator includes an electric circuit such as a transformer and a rectifier, and generates a high voltage to be applied to the X-ray tube 11 and a filament current to be supplied to the X-ray tube 11. The X-ray control device controls an output voltage according to the X-rays emitted from the X-ray tube 11. The high-voltage generator may be a transformer type or an inverter type. The X-ray high-voltage device 14 may be provided in the rotary frame 13 in the gantry 10, or may be provided in a fixed frame (not shown) in the gantry 10.

The wedge 16 adjusts the dose of the X-rays with which the subject P is irradiated. Specifically, the wedge 16 attenuates the X-rays so that the dose of the X-rays emitted from the X-ray tube 11 to the subject P have a predetermined distribution. For example, a metal plate made of aluminum or the like such as a wedge filter or a bow-tie filter is used as the wedge 16.

The collimator 17 limits an irradiation range of the X-rays transmitted through the wedge 16. The collimator 17 slidably supports a plurality of lead plates that shield X-rays, and adjusts a form of a slit formed by the plurality of lead plates. The collimator 17 may also be referred to as an X-ray aperture.

The data acquisition system 18 collects count data of X-rays detected by the X-ray detector 12 for each energy bin. As an example, the data acquisition system 18 includes a pre-amplifier, a waveform shaping circuit, a pulse-height discriminator circuit, and a counting circuit. The pre-amplifier amplifies an electric signal having a peak value corresponding to the energy of the X-ray photon detected by the X-ray detector 12 at a predetermined magnification. The waveform shaping circuit shapes the waveform of the electrical signal output by the pre-amplifier. The pulse-height discriminator applies an energy threshold corresponding to each of the plurality of energy bins to the electrical signal output by the waveform shaping circuit, and outputs an electrical pulse signal corresponding to the energy bin to which the electrical signal belongs. The counting circuit counts the electrical pulse signal output from the pulse-height discriminator in units of view periods for each energy bin, thereby generating count data representing the number of the count of X-ray photons for each energy bin. The data acquisition system 18 is implemented by, for example, an application specific integrated circuit (ASIC). Digital data is transmitted to the console 40 via a non-contact data transmission device or the like.

The control device 15 controls the X-ray high-voltage device 14 and the data acquisition system 18 to perform a PCCT scan according to the control of a processing circuitry 45 of the console 40. The control device 15 includes a processing circuitry including a central processing unit (CPU), a micro processing unit (MPU), or the like, and a drive mechanism such as a motor and an actuator. The processing circuitry includes a processor such as a CPU and a memory such as a read only memory (ROM) and a random-access memory (RAN) as hardware resources. Furthermore, the control device 15 may be implemented by an ASIC or a field programmable gate array (FPGA). Furthermore, the control device 15 may be implemented by another complex programmable logic device (CPLD) or a simple programmable logic device (SPLD). The control device 15 has a function of controlling the operation of the gantry 10 and the couch 30 in response to an input signal from an input interface 43 (described later) attached to the console 40 or the gantry 10. For example, the control device 15 performs control to rotate the rotary frame 13 in response to an input signal, control to tilt the gantry 10, and control to operate the couch 30 and the top plate 33. Note that the control of tilting the gantry 10 is implemented by the control device 15 rotating the rotary frame 13 about an axis parallel to the X-axis direction based on the inclination angle (tilt angle) information input by the input interface attached to the gantry 10. Note that the control device 15 may be provided on the gantry 10 or may be provided on the console 40.

The couch 30 includes a base 31, a support frame 32, a top plate 33, and a couch driving device 34. The base 31 is installed on a floor surface. The base 31 is a housing that supports the support frame 32 so as to be movable in a direction perpendicular to the floor surface (Y-axis direction). The support frame 32 is a frame provided above the base 31. The support frame 32 slidably supports the top plate 33 along the rotation axis (Z axis). The top plate 33 is a flexible plate on which the subject P is placed.

The couch driving device 34 is accommodated in a housing of the couch 30. The couch driving device 34 is a motor or an actuator that generates power for moving the support frame 32 and the top plate 33 on which the subject P is placed. The couch driving device 34 operates according to control by the console 40 or the like.

The console 40 includes a memory 41, a display 42, an input interface 43, a communication interface 44, and a processing circuitry 45. Data communication among the memory 41, the display 42, the input interface 43, the communication interface 44, and the processing circuitry 45 is performed via a bus (BUS). Although the console 40 will be described as a separate body from the gantry 10, the gantry 10 may include a part of each component of the console 40 or the console 40.

The memory 41 is a storage device such as a hard disk drive (HDD), a solid-state drive (SSD), or an integrated circuit storage device that stores various types of information. The memory 41 stores, for example, count data and PCCT image data. The memory 41 may be a portable storage medium such as a compact disc (CD), a digital versatile disc (DVD), or a flash memory, in addition to an HDD, an SSD, or the like. The memory 41 may be a drive device that reads and writes various types of information from and to a semiconductor memory element such as a flash memory or a random-access memory (RAN). In addition, the storage area of the memory 41 may be in the X-ray computed tomography apparatus 1 or in an external storage device connected via a network. The memory 41 stores a database to be described later.

The display 42 displays various types of information. For example, the display 42 outputs the PCCT image generated by the processing circuitry 45, a graphical user interface (GUI) for receiving various operations from the operator, and the like. As the display 42, any of various types of displays can be used as appropriate. For example, a liquid crystal display (LCD), a cathode ray tube (CRT) display, an organic electroluminescence (OELD) display, or a plasma display can be used as the display 42. Furthermore, the display 42 may be provided on the gantry 10. In addition, the display 42 may be a desktop type or may be configured by a tablet terminal or the like capable of wirelessly communicating with the main body of the console 40.

The input interface 43 receives various input operations from an operator, converts the received input operations into electrical signals, and outputs the electrical signals to the processing circuitry 45. As the input interface 43, for example, a mouse, a keyboard, a trackball, a switch, a button, a joystick, a touch pad, a touch panel display, and the like can be appropriately used. Note that, in the present embodiment, the input interface 43 is not limited to one including physical operation components such as a mouse, a keyboard, a trackball, a switch, a button, a joystick, a touch pad, and a touch panel display. For example, an electric signal processing circuitry that receives an electric signal corresponding to an input operation from an external input device provided separately from the device and outputs the electric signal to the processing circuitry 45 is also included in the example of the input interface 43. Furthermore, the input interface 43 may be provided on the gantry 10. In addition, the input interface 43 may be configured by a tablet terminal or the like capable of wirelessly communicating with the main body of the console 40.

The communication interface 44 includes a network interface card (NIC) or the like for communicating various data with an external device such as a workstation, a picture archiving and communication system (PACS), a radiology information system (RIS), or a hospital information system (HIS) via a network.

The processing circuitry 45 controls the entire operation of the X-ray computed tomography apparatus 1 according to the electric signal of the input operation output from the input interface 43. The processing circuitry 45 includes a processor such as a CPU and a memory such as a ROM and a RAM as hardware resources. The processing circuitry 45 implements various functions by a processor that executes a program expanded in a memory. The various functions are not limited to being implemented by a single processing circuitry. A processing circuitry may be configured by combining a plurality of independent processors, and each processor may execute a program to implement various functions.

As shown in FIG. 1, the processing circuitry 45 implements a scan control function 51, an acquisition function 52, a reconstruction function 53, a utilization information generation function 54, a comment input function 55, a storage function 56, and a display control function 57.

In the scan control function 51, the processing circuitry 45 controls the gantry 10 to perform the PCCT scan on the subject P. Under the control of the processing circuitry 45, the PCCT scan is performed by the gantry 10. With the PCCT scan performed, the data acquisition system 18 collects count data for each of the plurality of energy bins for each view.

In the acquisition function 52, the processing circuitry 45 acquires various types of information. As an example, the processing circuitry 45 may obtain count data related to a plurality of energy bins, which is collected by a PCCT scan on the subject P, from the data acquisition system 18. If count data is stored in the memory 41, the processing circuitry 45 may obtain the count data related to the plurality of energy bins from the memory 41.

In the reconstruction function 53, the processing circuitry 45 reconstructs one or a plurality of PCCT images based on count data related to a first energy bin among the plurality of energy bins. The PCCT image means an image based on the count data collected by the PCCT scan.

The PCCT image according to the present embodiment may be any one of a two-dimensional image related to one slice or slab and a three-dimensional image related to one volume. A two-dimensional image means image data including a plurality of pixels (pixels) arranged in a two-dimensional space, and a three-dimensional image means image data including a plurality of pixels (voxels) arranged in a three-dimensional space. Hereinafter, it is assumed that the PCCT image is a three-dimensional image.

In the utilization information generation function 54, the processing circuitry 45 generates information for evaluating a utilization degree of X-ray energy of an X-ray emitted in the PCCT scan (hereinafter, energy utilization information) for reconstruction of the one or plurality of PCCT images based on the first energy bin used to reconstruct the one or plurality of PCCT images or a second energy bin not used to reconstruct the one or plurality of PCCT images other than the first energy bin of the plurality of energy bins. The first energy bin means the energy bin contributing to imaging and the second energy bin means the energy bin not contributing to imaging. The energy utilization information specifically includes an identifier and/or an energy range of the first energy bin or the second energy bin, an energy utilization efficiency representing a ratio of the energy range of the first energy bin to the energy ranges of the plurality of energy bins, and/or an energy non-utilization efficiency representing a ratio of the energy range of the second energy bin to the energy ranges of the plurality of energy bins.

In the comment input function 55, the processing circuitry 45 inputs a comment with respect to the energy utilization information generated by the utilization information generation function 54. As an example, the processing circuitry 45 may input a comment only if the utilization degree indicated by the energy utilization information generated by the utilization information generation function 54 does not satisfy a predetermined condition. The condition is defined as that the energy utilization information has reached a level sufficient to be evaluated as appropriate. This condition is referred to as an appropriate condition. The processing circuitry 45 inputs a comment in a free input format or a format selected from fixed phrases.

In the storage function 56, the processing circuitry 45 stores the energy utilization information generated by the utilization information generation function 54 in the storage device in association with one or more PCCT images and/or related information of the one or plurality of PCCT images. The related information is, for example, DICOM files of one or a plurality of PCCT images, performed scan information related to a PCCT scan for collecting count data to be used for the one or the plurality of PCCT images, and/or order information for the PCCT scan. The storage device may be the memory 41 provided in the X-ray computed tomography apparatus 1, or may be a storage device in an information system such as PACS, HIS, or RIS connected via the communication interface 44. Hereinafter, the storage device in which the energy utilization information is stored is referred to as a data storage device. The data storage device is assumed to be the memory 41 provided in the X-ray computed tomography apparatus 1 unless otherwise specified.

In the display control function 57, the processing circuitry 45 displays various types of information on the display 42. As an example, the processing circuitry 45 displays one or a plurality of PCCT images reconstructed by the reconstruction function 53, energy utilization information generated by the utilization information generation function 54, and the like. At this time, the processing circuitry 45 may display the energy utilization information together with one or a plurality of PCCT images. Note that the processing circuitry 45 converts the PCCT image into a visualized image and displays the visualized image on the display 42. As the conversion processing to the visualized image, conversion processing from a three-dimensional image to a two-dimensional image, such as a pixel value projection method such as maximum intensity projection (MIP), multi-planar reconstruction (MPR), volume rendering, or surface rendering, is used.

Hereinafter, details of the X-ray computed tomography apparatus 1 will be described. In the following description, the first energy bin is also referred to as a used bin, and the second energy bin is also referred to as an unused bin.

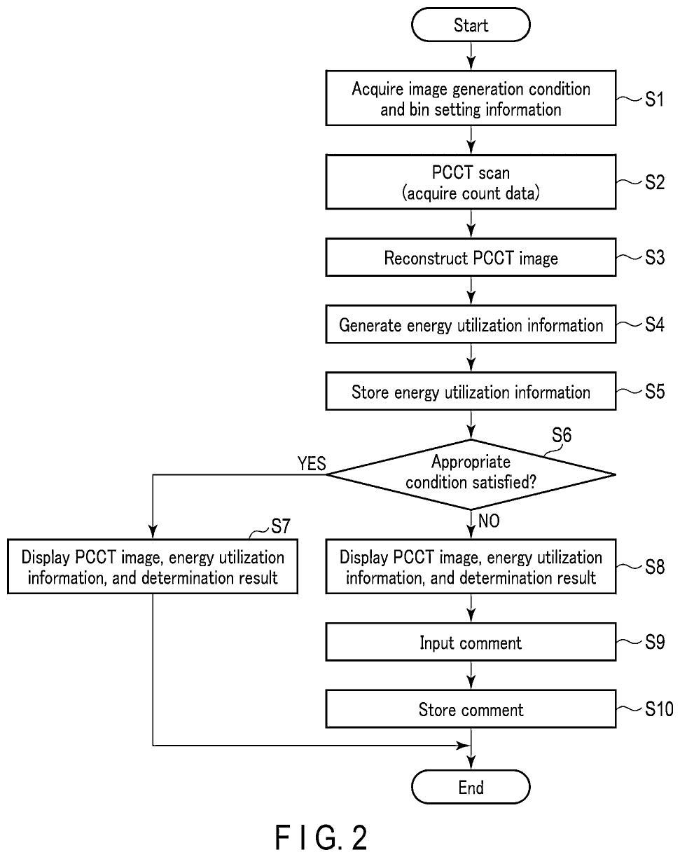

FIG. 2 is a view showing an example of a process procedure of a PCCT examination by the X-ray computed tomography apparatus 1. As shown in FIG. 2, first, the processing circuitry 45 acquires the image generation condition and the bin setting information by the acquisition function 52 (step S1). The image generation condition and the bin setting information are set before the PCCT scan is performed. For example, the image generation condition and the bin setting information are set as one piece of content of the protocol information by the RIS. The protocol information is information indicating a processing procedure of the PCCT examination, a setting condition of each step, and the like, and includes a scan condition of the PCCT scan and the like, in addition to the image generation condition and the bin setting information. The RIS sets protocol information based on the order information of the PCCT examination. In step S1, the processing circuitry 45 acquires protocol information from the RIS. As a result, the processing circuitry 45 acquires the image generation condition and the bin setting information. Note that the image generation condition and the bin setting information may be set by another hospital information system, the X-ray computed tomography apparatus 1, or the like. The acquired image generation condition and bin setting information are stored in the memory 41.

The image generation condition is a condition item related to the reconstruction of the PCCT image, and specifically includes the examination region of the subject P and the image generation purpose. As the examination region, any body part of the subject P such as a head, a chest, an abdomen, a leg, a brain, a heart, a lung, and a liver can be designated. As the image generation purpose, any type of PCCT image that can be generated from count data, such as an energy integral image, a blood vessel emphasis or suppression image, a fat emphasis or suppression image, a calcium emphasis or suppression image, an iodine emphasis or suppression image, a uric acid emphasis or suppression image, a contrast emphasis image, an electron density image, an effective atomic number image, a virtual monochromatic image, a basis material image, and/or a K-edge image, may be specified. The type of PCCT image may be specified along with the used bin and the X-ray energy. For example, an energy integral image using the second and third energy bins, a virtual monochromatic image of 70 keV, and the like may be specified.

The bin setting information means information about setting of the energy bins, and has, as items thereof, the number of energy bins, and the energy range, the energy width, and used/unused differentiation information of each energy bin. The used/unused differentiation information is a label indicating whether or not each energy bin is used for reconstruction of a PCCT image according to an image generation purpose. Specifically, the label “used” is used as the used/unused differentiation information indicating the energy bin to be used for the reconstruction of the PCCT image, and the label “non-used” is used as the use/non-use differentiation information indicating the energy bin not to be used for the reconstruction of the PCCT image.

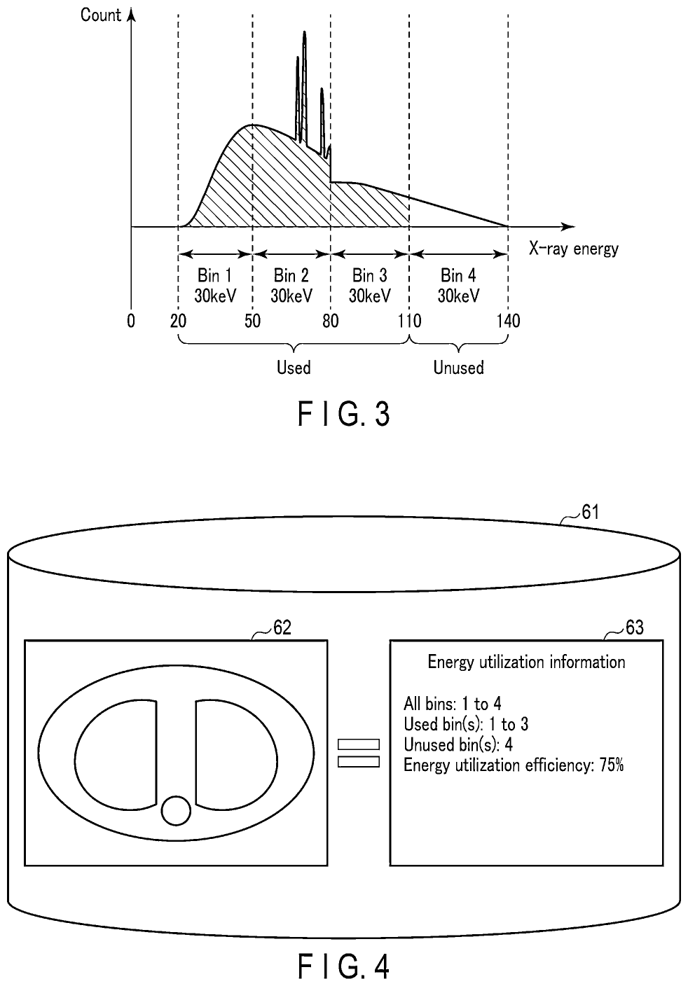

FIG. 3 is a diagram showing an example of setting bin setting information. FIG. 3 shows a relationship between an energy spectrum and an energy bin of X-rays emitted from the X-ray tube 11. The vertical axis of the energy spectrum in FIG. 3 represents counts, and the horizontal axis represents X-ray energy.

In FIG. 3, the tube voltage value is assumed as “140 kVp”, and the image generation purpose is assumed as “energy integral image using first to third energy bins”. In addition, the bin setting information is assumed as follows. In the following description, the energy bin may also be simply referred to as a bin. The Xth energy bin may also be simply referred to as bin X.
Number of energy bins=4
Bin 1: energy range=20 to 50 keV, energy width=30 keV, used/unused differentiation information=“used”
Bin 2: energy range=50 to 80 keV, energy width=30 keV, used/unused differentiation information=“used”
Bin 3: energy range=80 to 110 keV, energy width=30 keV, used/unused differentiation information=“used”
Bin 4: energy range=110 to 140 keV, energy width=30 keV, used/unused differentiation information=“unused”

Once step S1 is performed, the processing circuitry 45 executes a PCCT scan on the subject P by the scan control function 51 (step S2). In step S2, the processing circuitry 45 controls the gantry 10 according to a separately set scan condition to execute the PCCT scan on the subject P. Specifically, at first, the number and value of the energy thresholds of the data acquisition system 18 are set according to the number and energy range of the energy bins included in the bin setting information. The control device 15 controls the X-ray high-voltage device 14 to emit the X-rays from the X-ray tube 11 while rotating the rotary frame 13 at a high speed. The X-ray detector 12 detects the X-rays emitted from the X-ray tube 11 and transmitted through the subject P. The data acquisition system 18 collects count data for each of the plurality of energy bins for each view via the X-ray detector 12. The count data is transmitted to the console 40. The processing circuitry 45 acquires count data by the acquisition function 52.

Once step S2 is performed, the processing circuitry 45 reconstructs the PCCT image, by the reconstruction function 53, based on the count data acquired in step S2 (step S3). In step S3, the processing circuitry 45 specifically reads the image generation purpose and the bin setting information stored in the memory 41. Next, the processing circuitry 45 reads the used/unused differentiation information in the bin setting information, and selects the count data of the used bin from the count data acquired in step S2. Next, the processing circuitry 45 reconstructs the PCCT image according to the image generation purpose based on the selected count data. In the present embodiment, it is assumed that one PCCT image is reconstructed.

Once step S3 is performed, the processing circuitry 45 generates energy utilization information by the utilization information generation function 54 (step S4). In step S4, the processing circuitry 45 generates energy utilization information based on the bin setting information acquired in step S1. Specifically, the processing circuitry 45 extracts information about used bins and/or unused bins from the bin setting information, such as numbers of used bins and/or unused bins, energy ranges, and/or energy widths. The processing circuitry 45 then generates used/unused bin information representing identifiers and/or energy ranges for the used bins and/or unused bins, and energy utilization efficiency representing a ratio of the energy width of the used bins to the energy width of all bins. Hereinafter, processing of generating the used/unused bin information and the energy utilization efficiency will be described.

The used/unused bin information: the identifier, the energy range and/or the energy width for the used bins and/or unused bins are recorded in the bin setting information, so that the processing circuitry 45 can extract the identifier, the energy range and/or the energy width for the used bins and/or unused bins from the bin setting information. If the bin setting information does not include the energy width, the processing circuitry 45 may calculate the energy width from the energy range. In addition, if the energy range is defined by the lower limit value and the upper limit value of the energy range, the processing circuitry 45 may set a range between the upper limit value and the lower limit value as the energy range, and calculate the energy width by subtracting the lower limit value from the upper limit value. By comparing the identifiers and/or energy ranges of all the bins with the identifiers and/or energy ranges for the used bins and/or unused bins, a user such as a doctor or technician can gauge the degree to which the energy of the irradiated X-rays is utilized for reconstruction of the PCCT image. Therefore, the identifier and/or the energy range for the used bins and/or unused bins can be said to be a kind of energy utilization information.

Energy utilization efficiency: The processing circuitry 45 calculates the energy utilization efficiency based on the bin setting information. Specifically, the processing circuitry 45 first specifies the energy width of all the bins and the energy width of the used bins from the bin setting information, and divides the energy width of the used bins by the energy width of all the bins to calculate the energy utilization efficiency that is the ratio of the energy width of the used bins to the energy width of all the bins. Since the energy utilization efficiency represents the ratio of the energy range of the X-ray energy contributing to imaging to the energy range of the irradiated X-rays, the user can estimate the degree to which the energy of the irradiated X-rays is utilized for the reconstruction of the PCCT image by confirming the energy utilization efficiency. Therefore, it can be said that the energy utilization efficiency is a kind of energy utilization information.

Once step S4 is performed, the processing circuitry 45 stores the energy utilization information generated in step S4 in the data storage device by the storage function 56 (step S5). In step S5, the processing circuitry 45 stores the energy utilization information in the data storage device in association with the PCCT image reconstructed in step S3.

FIG. 4 is a conceptual diagram of saving a PCCT image 62 and energy utilization information 63 in a data storage device 61. The bin setting information in the case of FIG. 4 assumes bin setting information similar to that of FIG. 3. As shown in FIG. 4, the energy utilization information 63 includes numbers of all bins, numbers of used bins, numbers of unused bins, and energy utilization efficiency. Specifically, all bins are bins 1 to 4, used bins are bins 1 to 3, and an unused bin is a bin 4. The energy width for all bins is 140 keV-20 keV=120 keV because the energy range is 20 to 140 keV. The energy width for used bins is 110 keV-20 keV=90 keV because the energy range is 20 to 110 keV. Therefore, the energy utilization efficiency, which is the ratio of the energy width of the used bins to the energy width of all bins, is calculated to be 90 keV/120 keV=75%.

Once step S5 is performed, the processing circuitry 45 determines whether the appropriate condition is satisfied by the comment input function 55 (step S6). The appropriate condition may be set for the used bin or the unused bin, or may be set for the energy utilization efficiency. The appropriate condition for the used bin or the unused bin may be set such that the energy bin on the highest energy side among the plurality of energy bins is used, that an energy bin having energy equal to or higher than the threshold is used, or the like. The appropriate condition for the energy bin utilization efficiency may be set such that the energy utilization efficiency exceeds a threshold value. The appropriate condition is not limited to the above, and can be arbitrarily set.

Once it is determined in step S6 that the appropriate condition is satisfied (step S6: YES), the processing circuitry 45 displays the PCCT image reconstructed in step S3, the energy utilization information generated in step S4, and the determination result of step S6 on the display 42 by the display control function 57 (step S7).

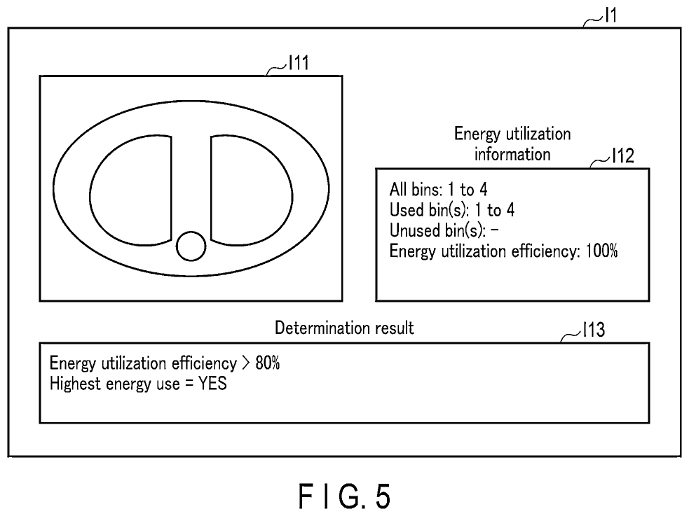

FIG. 5 is a diagram showing an example of a display screen I1 of a PCCT image I11, energy utilization information I12, and a determination result I13 according to step S7. The bin setting information in FIG. 5 is assumed as follows.
Number of energy bins=4
Bin 1: energy range=20 to 50 keV, energy width=30 keV, used/unused differentiation information=“used”
Bin 2: energy range=50 to 80 keV, energy width=30 keV, used/unused differentiation information=“used”
Bin 3: energy range=80 to 110 keV, energy width=30 keV, used/unused differentiation information=“used”
Bin 4: energy range=110 to 140 keV, energy width=30 keV, used/unused differentiation information=“used”

As noted above, all of the bins 1 to 4 are used bins, there are no unused bins, and the energy utilization efficiency is 100%. In addition, it is assumed that the appropriate conditions are two conditions of a first condition “energy utilization efficiency exceeds 80%” and a second condition “energy bin on the highest energy side is used”. In the bin setting information in FIG. 5, the energy utilization efficiency is 100%, which means exceeding 80%, so that the first condition is satisfied, and the used bins are the bins 1 to 4, and the bin 4 that is the energy bin on the highest energy side is used, so that the second condition is also satisfied.

As shown in FIG. 5, the PCCT image I11 reconstructed in step S3, the energy utilization information I12 generated in step S4, and the determination result I13 of step S6 are displayed side by side on the display screen I1. As the energy utilization information I12, texts such as “all bins: 1 to 4”, “used bins: 1 to 4”, “unused bins: -”, and “energy utilization efficiency: 100%” are displayed. As the determination result I13, a text indicating that the first condition is satisfied, such as “energy utilization efficiency>80%”, is preferably displayed as the determination result of the first condition, and a text indicating that the second condition is satisfied, such as “highest energy use=YES”, is preferably displayed as the determination result of the second condition.

Once step S7 is performed, the PCCT examination shown in FIG. 2 ends.

On the other hand, once it is determined in step S6 that the appropriate condition is not satisfied (step S6: NO), the processing circuitry 45 displays the PCCT image reconstructed in step S3, the energy utilization information generated in step S4, and the determination result of step S6 by the display control function 57 (step S8).

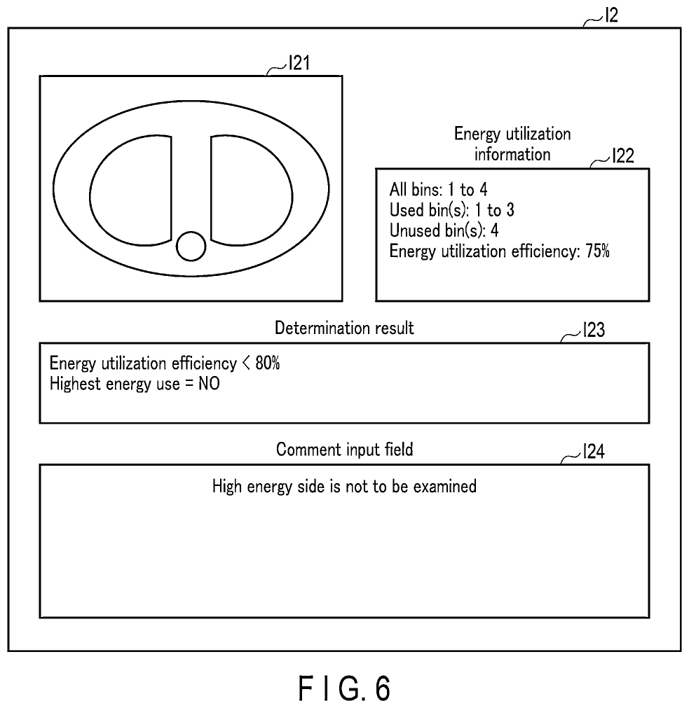

FIG. 6 is a diagram showing an example of a display screen I2 of a PCCT image I21, energy utilization information I22, and a determination result I23 according to step S8. The bin setting information in the case of FIG. 6 assumes bin setting information similar to that of FIG. 3. In addition, similarly to the case of FIG. 5, it is assumed that the appropriate conditions are two conditions of the first condition “energy utilization efficiency exceeds 80%” and the second condition “energy bin on the highest energy side is used”. In the case of FIG. 6, the energy utilization efficiency is 75%, which means not exceeding 80%, so that the first condition is not satisfied, and the used bins are the bins 1 to 3, and the bin 4 that is the energy bin on the highest energy side is not used, so that the second condition is not also satisfied.

As shown in FIG. 6, the PCCT image I21 reconstructed in step S3, the energy utilization information I22 generated in step S4, and the determination result I23 of step S6 are displayed side by side on the display screen I2. As the energy utilization information I22, texts such as “all bins: 1 to 4”, “used bins: 1 to 3”, “unused bins: 4”, and “energy utilization efficiency: 75%” are displayed. As the determination result I23, a text indicating that the first condition is not satisfied, such as “energy utilization efficiency<80%”, is preferably displayed as the determination result of the first condition, and a text indicating that the second condition is not satisfied, such as “highest energy use=NO”, is preferably displayed as the determination result of the second condition.

As illustrated in FIG. 6, if the appropriate condition is not satisfied, a comment input field I24 may be further displayed on the display screen I2. In step S9, a comment regarding a circumstance, a reason, an explanation, or the like regarding non-satisfaction of the appropriate condition is input to the comment input field I24. An input format of the comment into the comment input field I24 is assumed to be a free input format (free text format).

Once step S8 is performed, the processing circuitry 45 inputs a comment by the comment input function 55 (step S9). If the appropriate condition is not satisfied, the processing circuitry 45 prompts the user to input a comment regarding a circumstance, a reason, an explanation, or the like regarding non-satisfaction of the appropriate condition. For example, the processing circuitry 45 may display a message window such as “Please input a comment” in a superimposed manner on the display screen I2 shown in FIG. 6, may output speech such as “Please input a comment” via a speaker, or may emphasize the comment input field I24 with a visual effect such as blinking. Note that the processing circuitry 45 may prompt the user to input a comment by simply displaying the comment input field I24.

In step S9, the user freely inputs a comment such as “high energy side is not to be examined” to the comment input field I24 via the input interface 43. The text of the input comment is immediately displayed in the comment input field I24.

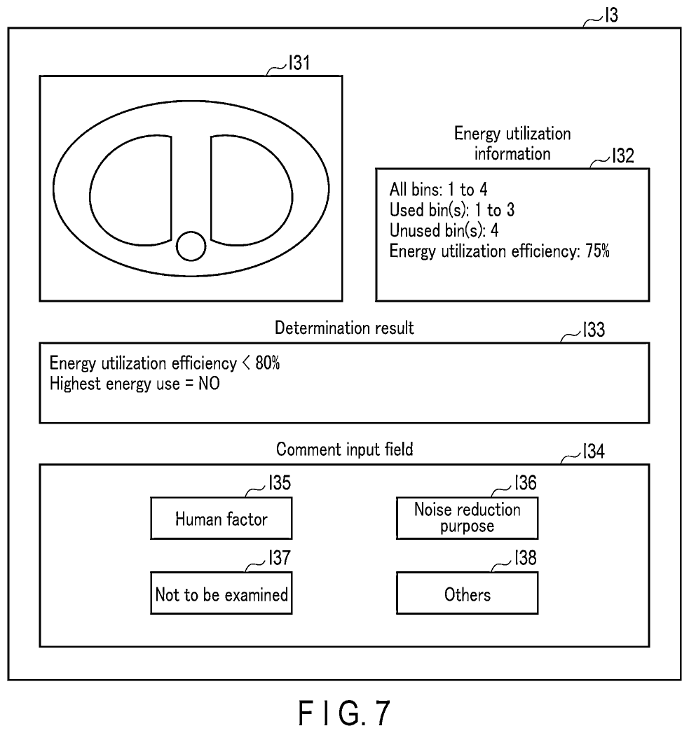

FIG. 7 is a diagram showing another example of a display screen I3 of the PCCT image I21, the energy utilization information I22, and the determination result I23 according to step S8. The case of FIG. 7 is the same as the case of FIG. 6. A PCCT image I31 reconstructed in step S3, energy utilization information I32 generated in step S4, and a determination result I33 of step S6 are displayed side by side on the display screen I3.

As illustrated in FIG. 7, a comment input field I34 of a selection formula is displayed on the display screen I3. Graphical User Interface (GUI) buttons I35 to I38 on which fixed phrases representing typical comment contents are printed are displayed in the comment input field I34. Once the GUI buttons I35 to I38 are pressed, text associated with the GUI buttons I35 to I38 is input. For example, once the GUI button I35 on which the fixed phrase “human factor” is printed is pressed, a comment indicating that the appropriate condition is not satisfied due to the human factor is input. As another example, once the GUI button I36 on which the fixed phrase “noise removal purpose” is printed is pressed, a comment indicating that the appropriate condition is not satisfied due to the energy range in which noise is dominant being set to the unused bin is input. As another example, once the GUI button I37 on which the fixed phrase “not to be examined” is printed is pressed, a comment indicating that the appropriate condition is not satisfied due to the energy range in which a tissue component not to be examined is dominant being set to the unused bin is input. As another example, once the GUI button I38 on which the fixed phrase “others” is printed is pressed, a free-form comment input field may be superimposed and displayed on the display screen I3. By filling the selection formula, it is possible to save time and effort for inputting a comment and to unify comments of similar factors.

Once step S9 is performed, the processing circuitry 45 stores the comment input in step S9 in the data storage device by the storage function 56 (step S10). In step S10, as an example, the processing circuitry 45 stores the comment in association with the PCCT image reconstructed in step S3 and the energy utilization information generated in step S4.

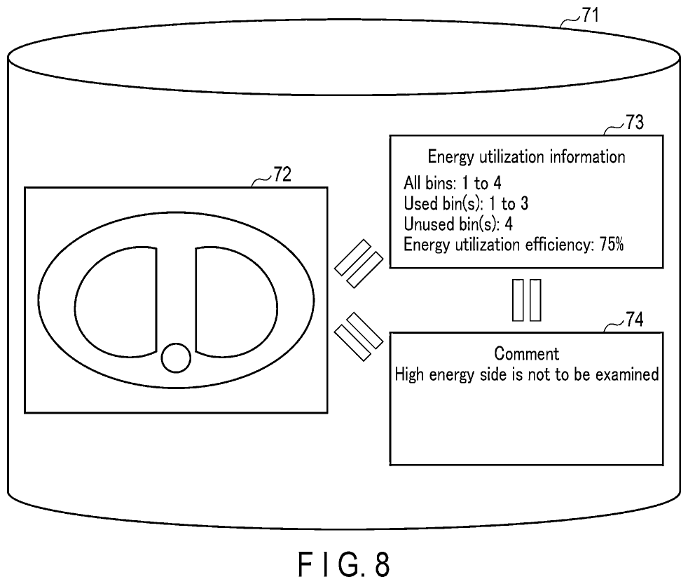

FIG. 8 is a conceptual diagram of saving a comment 74 in a data storage device 71. As shown in FIG. 8, the data storage device 71 further stores the comment 74 in addition to a PCCT image 72 and energy utilization information 73. The PCCT image 72, the energy utilization information 73, and the comment 74 may be stored in association with each other. As a result, by storing the comment 74, it is possible to record the utilization degree of the irradiated X-ray energy in the PCCT examination, specifically, the opinion of the implementer of the PCCT examination at the time of examination regarding the fact that the energy utilization information does not satisfy the appropriate condition. Furthermore, the comment 74 can be easily read by associating the comment 74 with the PCCT image 72 and the energy utilization information 73.

A storage mode of the comment 74 is not limited to the above. As an example, the comment 74 may be associated with only one of the PCCT image 72 and the energy utilization information 73. As another example, the comment 74 may be incorporated into the comment field of the PCCT image 72, or the comment 74 may be incorporated as the content of the energy utilization information 73.

Thus, the PCCT examination shown in FIG. 2 ends.

Note that the processing procedure of the PCCT examination shown in FIG. 2 is an example, and the present embodiment is not limited thereto. As an example, the determination of the appropriate condition (S6) and the subsequent processing (S7 to S10) are not essential, and steps S6 to S10 may be omitted. The determination of the appropriate condition (S6) is not essential, and the comment input (S9) and the saving (S10) may be performed unconditionally. Generation (S4) and storage (S5) of the energy utilization information may be performed between steps S1 and S2 or between steps S2 and S3. The storage of the energy utilization information (S5) may be performed after the display (S7 and/or S8).

The above embodiment is an example, and the present embodiment is not limited to the embodiment, and any element can be added, deleted, and/or changed without departing from the gist of the invention.

### First Modification

In the above embodiment, the processing circuitry 45 associates the energy utilization information with the PCCT image. However, the energy utilization information may be indirectly associated with the PCCT image. The processing circuitry 45 according to a first modification associates the energy utilization information with the related information of the PCCT image. Hereinafter, the first modification will be specifically described. Note that, in the following description, components having substantially the same functions as those of the above embodiment will be denoted by the same reference numerals, and redundant description will be given only when necessary.

The related information is information directly or indirectly associated with the PCCT image. Specifically, as the related information, a DICOM file of the PCCT image, performed scan information related to a PCCT scan for collecting count data used for reconstructing the PCCT image, and/or the order information for the PCCT scan can be used.

The DICOM file has a study file for each DICOM study. The DICOM file is stored in the data storage device. The data storage device may be the memory 41 of the X-ray computed tomography apparatus 1 or a storage device provided in a hospital information system such as a PACS, a RIS, or a HIS. The processing circuitry 45 stores the energy utilization information in the study file related to the PCCT scan of the DICOM study. Since the PCCT image is stored in the DICOM file, the energy utilization information can be associated with the PCCT image by storing the energy utilization information in the study file.

The performed scan information includes various types of information regarding the PCCT examination, such as radiation dose information, billing information, and material usage information regarding the performed PCCT scan. The radiation dose information is managed as a Radiation Dose Structured Report (RDSR) object in the DICOM standard. As an example, the processing circuitry 45 according to the first modification uses the energy utilization information as a piece of content of the radiation dose information among the performed scan information. Specifically, if the energy utilization information is generated, the processing circuitry 45 transmits the energy utilization information to the RIS according to a Modality Performed Procedure Step (MPPS). MPPS means a communication procedure defined in the DICOM standard. By transmitting the energy utilization information according to the MPPS, the RIS stores the energy utilization information in its own storage device as performed scan information of the DICOM specification, specifically, as an RDSR object. As a result, the energy utilization information can be stored in association with the performed scan information. Note that the processing circuitry 45 may store the energy utilization information in the memory 41 in association with the performed scan information.

The order information means the examination order of the performed PCCT scan. As an example, the order information is issued by the HIS and transmitted to the RIS. The RIS sets an image generation condition, bin setting information, and a scan condition based on the order information. The image generation condition, the bin setting information, and the scan condition are transmitted from the RIS to the X-ray computed tomography apparatus 1. If the energy utilization information is generated, the processing circuitry 45 transmits the energy utilization information to the RIS according to the MPPS. The RIS stores the energy utilization information in the data storage device in association with the order information. As a result, the energy utilization information can be stored in association with the order information. Note that the processing circuitry 45 may store the energy utilization information in the memory 41 in association with the order information.

### Second Modification

In the above embodiment, the medical information processing apparatus 40 is the console provided in the X-ray computed tomography apparatus 1. However, the present embodiment is not limited thereto. The medical information processing apparatus 40 according to the second modification may be a computer separate from the X-ray computed tomography apparatus 1 such as an image interpretation apparatus, quality assurance apparatus, or a workstation. In this case, the medical information processing apparatus 40 does not need to include the scan control function 51. The processing circuitry 45 according to the second modification may acquire count data from a hospital information system such as an X-ray computed tomography apparatus or a PACS, and may reconstruct a PCCT image, generate energy utilization information, and store the energy utilization information as in the above embodiment.

According to the second modification, in the medical information processing apparatus 40 independent of the X-ray computed tomography apparatus 1, it is possible to reconstruct the PCCT image, generate the energy utilization information, and store the energy utilization information, similarly to the above embodiment. As a result, it is possible to grasp the utilization degree of the X-ray energy related to the PCCT scan.

### Third Modification

The processing circuitry 45 according to the above embodiment calculates the ratio of the energy width of the first energy bin (used bin) used for reconstructing the PCCT image to the energy widths of the plurality of energy bins (all bins) as the energy utilization efficiency. However, the definition of the energy utilization efficiency is not limited to the above, and various modifications are possible. Which of the following energy utilization efficiencies is adopted can be arbitrarily selected by a user or the like via the input interface 43 or the like.

As an example, the processing circuitry 45 according to the third modification may set the ratio of the number of used bins to the number of all bins as the energy utilization efficiency. As another example, the processing circuitry 45 may calculate the ratio of the energy weighting energy width of the used bin to the energy weighting energy width of all bins as the energy utilization efficiency. Here, the energy weighting energy width of all bins or used bins is calculated as an integral value of the X-ray energy over the energy range to which all bins or used bins belong.

As another example, the processing circuitry 45 may calculate the energy utilization efficiency in consideration of the X-ray spectrum of the irradiated X-rays or the X-ray spectrum of the detected X-rays. In other words, the processing circuitry 45 may calculate the ratio of the energy weighting energy width of the used bin to the energy width weighted by the count and energy of all bins as the energy utilization efficiency. The counts and energy weighting energy widths for all or used bins are computed as an integral value of the X-ray spectrum for the energy range to which all or used bins belong.

### Fourth Modification

In the above embodiment, the processing circuitry 45 calculates the energy utilization efficiency for one PCCT image based on one piece of count data. However, the present embodiment is not limited thereto. The processing circuitry 45 according to the fourth modification calculates the energy utilization efficiency for a plurality of PCCT images based on one piece of count data. In this case, the processing circuitry 45 calculates the ratio of the energy width of the first energy bin (used bin) used for reconstruction of any one image of the plurality of PCCT images to the energy widths of the plurality of energy bins (all bins) as the energy utilization efficiency of the plurality of PCCT images. Hereinafter, the fourth modification will be specifically described. Note that, in the following description, components having substantially the same functions as those of the above embodiment will be denoted by the same reference numerals, and redundant description will be given only when necessary. In addition, energy utilization efficiency for one PCCT image is referred to as single energy utilization efficiency, energy utilization information for one PCCT image is referred to as single energy utilization information, energy utilization efficiency for a plurality of PCCT images is referred to as image group energy utilization efficiency, and energy utilization efficiency for a plurality of PCCT images is referred to as image group energy utilization information.

As a scene of reconstructing a plurality of PCCT images based on one piece of count data, a scene of reconstructing a plurality of PCCT images belonging to the same Digital Imaging and Communication in Medicine (DICOM) study is assumed as an example.

As an example, the bin setting information according to the fourth modification is assumed as follows.
Number of energy bins=4
Bin 1: energy range=20 to 50 keV, energy width=30 keV
Bin 2: energy range=50 to 80 keV, energy width=30 keV
Bin 3: energy range=80 to 110 keV, energy width=30 keV
Bin 4: energy range=110 to 140 keV, energy width=30 keV

In the fourth modification, three PCCT images are reconstructed based on one piece of count data. The used/unused differentiation information for each PCCT image is assumed as follows.
First PCCT image: bin 1=“used”, bin 2=“used”, bin 3=“unused”, bin 4=“unused”
Second PCCT image: bin 1=“used”, bin 2=“unused”, bin 3=“unused”, bin 4=“unused”
Third PCCT image: bin 1=“unused”, bin 2=“used”, bin 3=“unused”, bin 4=“unused”

As described above, the energy width of all bins is 140 keV-20 keV=120 keV because the energy range is 20 to 140 keV, and the energy width of the used bins is 30 keV+30 keV=60 keV because the used bins used to reconstruct any one image of the three PCCT images are bins 1 and 2. Therefore, the image group energy utilization efficiency, which is the ratio of the energy width of the used bin used to reconstruct any one of the three PCCT images to the energy width of all the bins, is calculated to be 60 keV/120 keV=50%.

The processing circuitry 45 according to the fourth modification stores the image group energy utilization information in the data storage device in association with the plurality of PCCT images. The image group energy utilization information may be associated with each of the plurality of PCCT images or may be associated with related information of the plurality of PCCT images. As the related information, for example, a DICOM file in which the plurality of PCCT images is recorded can be used.

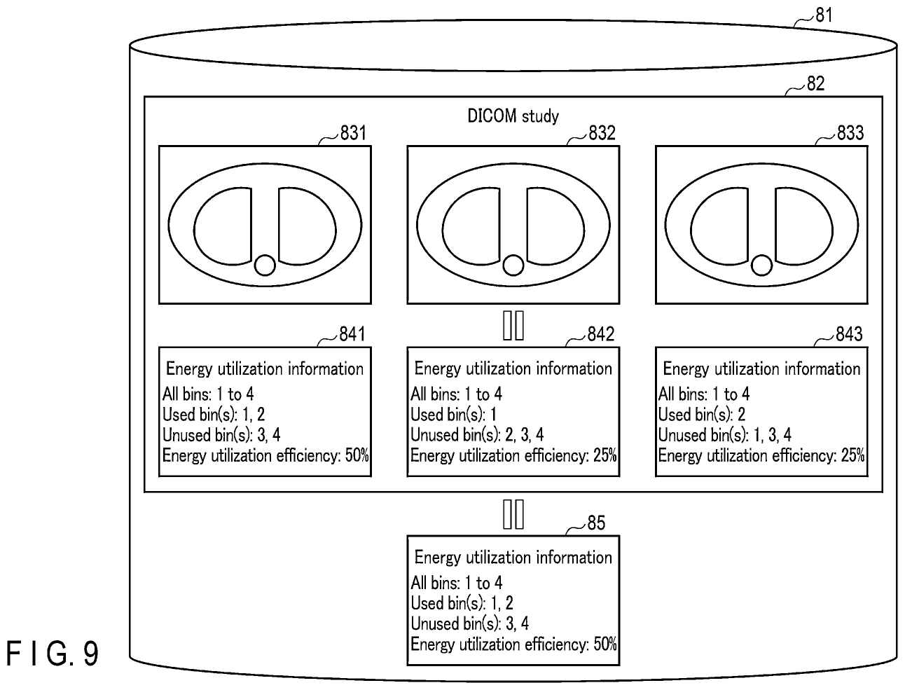

FIG. 9 is a conceptual diagram of saving energy utilization information 85 of a plurality of PCCT images 83n in a data storage device 81. As shown in FIG. 9, the three PCCT images 83n (n is a subscript of the PCCT image. n=1, 2, or 3) are associated with three pieces of single energy utilization information 84m (m is a subscript of the energy utilization information. m=1, 2 or 3), respectively. The energy utilization information 84m of the PCCT image 83n includes the single energy utilization efficiency generated by the above several methods. The image group energy utilization information 85 common to the three PCCT images 83n includes the image group energy utilization efficiency. While the single energy utilization efficiency 84m evaluates the utilization degree of energy of the irradiated X-rays for the corresponding PCCT image 83n, the image group energy utilization efficiency can evaluate the utilization degree of energy of the irradiated X-rays for the entire one DICOM study.

As shown in FIG. 9, the three PCCT images 83n, the three pieces of single energy utilization information 84m, and the image group energy utilization information 85 are recorded in a DICOM file 82. The DICOM file 82 is stored in the data storage device 81. The DICOM file 82 is a file that manages an image group associated with one PCCT examination, and specifically, manages three PCCT images reconstructed based on one piece of count data collected by a PCCT scan performed in one PCCT examination. By registering the energy utilization information 85 in the DICOM file 82, it is possible to associate the image group energy utilization information 85 with the three PCCT images 83n and the three pieces of single energy utilization information 84m. This makes it possible to manage the image group energy utilization information 85 over the three PCCT images 83n.

### Fifth Modification

As shown in FIG. 2 of the above embodiment, the processing circuitry 45 acquires the image generation condition and the bin setting information (S1) before performing the PCCT scan (S2). Therefore, the processing circuitry 45 can determine the presence or absence of the unused bin based on the bin setting information before performing the PCCT scan. The processing circuitry 45 according to a fifth modification may recommend the use of an X-ray attenuation filter that shields X-rays in an energy range to which the unused bin belongs in a case where the unused bin is present. The X-ray attenuation filter may constitute a part of the wedge 16 or the collimator 17, or may be a filter separate from the wedge 16 or the collimator 17. Hereinafter, the fifth modification will be specifically described. Note that, in the following description, components having substantially the same functions as those of the above embodiment will be denoted by the same reference numerals, and redundant description will be given only when necessary.

As an example, the bin setting information according to the fifth modification is assumed as follows.
Number of energy bins=4
Bin 1: energy range=20 to 50 keV, energy width=30 keV, “unused”
Bin 2: energy range=50 to 80 keV, energy width=30 keV, “unused”
Bin 3: energy range=80 to 110 keV, energy width=30 keV, “used”
Bin 4: energy range=110 to 140 keV, energy width=30 keV, “used”

In this case, the processing circuitry 45 recommends the use of an X-ray attenuation filter that shields X-ray energy equal to or less than the upper limit value 80 keV of the X-ray energy of the unused bin. The recommended method is not particularly limited, and for example, a message to recommend the X-ray attenuation filter may be displayed on the display 42, or a sound to recommend the X-ray attenuation filter may be output via a speaker. The user who has received the recommendation determines whether or not the use of the X-ray attenuation filter is necessary, and sets the use of the X-ray attenuation filter if the user determines that the use of the X-ray attenuation filter is necessary. This makes it possible to reduce irradiation of the subject with X-rays of X-ray energy components that do not contribute to imaging.

### Sixth Modification

In the above embodiment, the processing circuitry 45 calculates the energy utilization efficiency indicating the ratio of the energy range of the first energy bin (used bin) to the energy ranges of the plurality of energy bins (all bins) as one of the pieces of energy utilization information. However, the present embodiment is not limited thereto. The processing circuitry 45 according to a sixth modification calculates the energy non-utilization efficiency indicating the ratio of the energy range of the second energy bin (unused bin) to the energy ranges of the plurality of energy bins (all bins) as one of the pieces of energy utilization information. Hereinafter, the sixth modification will be specifically described. Note that, in the following description, components having substantially the same functions as those of the above embodiment will be denoted by the same reference numerals, and redundant description will be given only when necessary.

As an example, it is assumed that the bin setting information according to the sixth modification is the same as the bin setting information of the case of FIG. 3. That is, bins 1 to 3 are used bins and a bin 4 is an unused bin. In this case, the energy width of all bins is 140 keV-20 keV=120 keV because the energy range is 20 to 140 keV, and the energy width of the unused bin is 140 keV-110 keV=30 keV because the energy range is 110 to 140 keV. Therefore, the energy non-utilization efficiency, which is the ratio of the energy width of the unused bins to the energy width of all bins, is calculated to be 30 keV/120 keV=25%. That is, it means that 25% of the total energy does not contribute to imaging. The processing circuitry 45 can use the energy non-utilization efficiency as energy utilization information instead of or together with the energy utilization efficiency. For example, the processing circuitry 45 may store the energy non-utilization efficiency in association with the PCCT image and/or related information, or display the energy non-utilization efficiency on the display 42.

### Seventh Modification

In the above embodiment, the energy utilization information is generated at the time of performing the PCCT examination. In this case, the processing circuitry 45 is assumed to extract information regarding the first energy bin (used bin) and/or the second energy bin (unused bin) from the bin setting information set before performing the PCCT scan. However, the present embodiment is not limited thereto. It is also assumed that the PCCT image is reconstructed according to a new image generation condition and bin setting information based on the collected count data after the end of the PCCT examination. The processing circuitry 45 according to a seventh modification may also generate the energy utilization information for the PCCT image generated after the end of the PCCT examination. In this case, the processing circuitry 45 may set information regarding the first energy bin (used bin) and/or the second energy bin (unused bin) after performing the PCCT scan.

### CONCLUSION

As described above, the medical information processing apparatus 40 includes the processing circuitry 45 that implements the acquisition function 52, the reconstruction function 53, the utilization information generation function 54, and the storage function 56. The processing circuitry 45 acquires count data related to a plurality of energy bins collected in the PCCT scan on the subject P. The processing circuitry 45 reconstructs one or a plurality of PCCT images based on count data related to the first energy bin among the plurality of energy bins. The processing circuitry 45 generates energy utilization information for evaluating a utilization degree of X-ray energy of an X-ray emitted in the PCCT scan for reconstruction of the one or plurality of PCCT images based on the first energy bin used to reconstruct the one or plurality of PCCT images or a second energy bin not used to reconstruct the one or plurality of PCCT images other than the first energy bin of the plurality of energy bins. The processing circuitry 45 stores the energy utilization information in a data storage device in association with one or a plurality of PCCT images and/or related information of the one or plurality of PCCT images.

According to the above configuration, the energy utilization information can be generated as the index for evaluating the utilization degree of the irradiated X-ray energy related to the reconstruction of the PCCT image. By checking the energy utilization information, the medical worker engaged in the PCCT examination can grasp whether or not the irradiated X-ray energy is effectively utilized, the degree thereof, and the like. Accordingly, it can be expected to motivate the medical worker to increase the utilization degree of the irradiated X-ray energy or to suppress the generation of the irradiated X-ray energy that does not contribute to imaging. According to the above configuration, the energy utilization information can be stored for a long period of time in association with the PCCT image and/or the related information. As a result, it is also possible to check and verify the energy utilization information at any time after reading it from the data storage device.

According to at least one embodiment described above, the utilization degree of the irradiated X-ray energy related to the PCCT scan can be grasped.

The term “processor” used in the above description means, for example, a CPU, a GPU, or a circuit such as an application specific integrated circuit (ASIC) or a programmable logic device (for example, a simple programmable logic device (SPLD), a complex programmable logic device (CPLD), and a field programmable gate array (FPGA)). The processor implements a function by reading and executing a program stored in the storage circuit. Instead of storing the program in the storage circuit, the program may be directly incorporated in the circuit of the processor. In this case, the processor implements the function by reading and executing the program incorporated in the circuit. On the other hand, in a case where the processor is, for example, an ASIC, the function is directly incorporated as a logic circuit in a circuit of the processor instead of storing the program in the storage circuit. Note that each processor of the present embodiment is not limited to a case where each processor is configured as a single circuit, and a plurality of independent circuits may be combined and configured as one processor to implement the function. Furthermore, a plurality of components in FIG. 1 may be integrated into one processor to implement the function.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising a processing circuitry configured to
acquire count data related to a plurality of energy bins, which is collected in a PCCT scan on a subject;
reconstruct one or a plurality of PCCT images based on count data related to a first energy bin of the energy bins;
generate utilization information for evaluating a utilization degree of X-ray energy of X-rays emitted in the PCCT scan for reconstruction of the one or plurality of PCCT images based on the first energy bin used to reconstruct the one or plurality of PCCT images or a second energy bin not used to reconstruct the one or plurality of PCCT images other than the first energy bin of the energy bins; and
store the utilization information in a storage device in association with the one or plurality of PCCT images and/or related information of the one or plurality of PCCT images.

2. The medical information processing apparatus according to claim 1, wherein
the energy utilization information includes:
an identifier and/or an energy range of the first energy bin or the second energy bin;
an energy utilization efficiency representing a ratio of the energy range of the first energy bin to the energy ranges of the energy bins; and/or
an energy non-utilization efficiency representing a ratio of the energy range of the second energy bin to the energy ranges of the energy bins.

3. The medical information processing apparatus according to claim 2, wherein the processing circuitry is configured to calculate a ratio of an energy width of the first energy bin used for reconstructing the one PCCT image to energy widths of the energy bins as the energy utilization efficiency of the one PCCT image.

4. The medical information processing apparatus according to claim 2, wherein the processing circuitry is configured to calculate a ratio of an energy width of the first energy bin used for reconstructing any one of the plurality of PCCT images to energy widths of the energy bins as the energy utilization efficiency of the plurality of PCCT images.

5. The medical information processing apparatus according to claim 1, wherein
the processing circuitry is configured to:
input a comment with respect to the utilization information; and
store the comment in the storage device together with the utilization information.

6. The medical information processing apparatus according to claim 5, wherein the processing circuitry is configured to input the comment in a case where the utilization degree does not satisfy a predetermined condition.

7. The medical information processing apparatus according to claim 5, wherein the processing circuitry is configured to input the comment in a free input format or a format selected from fixed phrases.

8. The medical information processing apparatus according to claim 1, wherein the processing circuitry is configured to display the utilization information on a display device.

9. The medical information processing apparatus according to claim 8, wherein the display device displays the utilization information together with the one or plurality of PCCT images.

10. The medical information processing apparatus according to claim 1, wherein the processing circuitry is configured to extract information related to the first energy bin and/or the second energy bin from bin setting information set before performing the PCCT scan.

11. The medical information processing apparatus according to claim 1, wherein the processing circuitry is configured to set information related to the first energy bin and/or the second energy bin after performing the PCCT scan.

12. The medical information processing apparatus according to claim 1, wherein the related information is DICOM files of the one or plurality of PCCT images, performed scan information related to the PCCT scan and/or order information for the PCCT scan.

13. A medical information processing method comprising the steps of:
acquiring count data related to a plurality of energy bins, which is collected in a PCCT scan on a subject;
reconstructing one or a plurality of PCCT images based on count data related to a first energy bin of the energy bins;
generating utilization information for evaluating a utilization degree of X-ray energy of X-rays emitted in the PCCT scan for reconstruction of the one or plurality of PCCT images based on the first energy bin used to reconstruct the one or plurality of PCCT images or a second energy bin not used to reconstruct the one or plurality of PCCT images other than the first energy bin of the plurality of energy bins; and
storing the utilization information in a storage device in association with the one or plurality of PCCT images and/or related information of the one or plurality of PCCT images.

14. A non-transitory computer-readable storage medium storing a program that causes a computer, when interpreted and executed by the computer, to perform the steps of:
acquiring count data related to a plurality of energy bins, which is collected in a PCCT scan on a subject;
reconstructing one or a plurality of PCCT images based on count data related to a first energy bin of the plurality of energy bins;
generating utilization information for evaluating a utilization degree of X-ray energy of an X-ray emitted in the PCCT scan for reconstruction of the one or plurality of PCCT images based on the first energy bin used to reconstruct the one or plurality of PCCT images or a second energy bin not used to reconstruct the one or plurality of PCCT images other than the first energy bin of the plurality of energy bins; and
storing the utilization information in a storage device in association with the one or plurality of PCCT images and/or related information of the one or plurality of PCCT images.
